(19) United States
(12) Patent Application Publication
(10) Pub. No.: US 2016/0193302 A1
(43) Pub. Date: Jul. 7, 2016

(54) LACTOFERRIN AND MEMORY AND LEARNING SPEED IN CHILDREN

(71) Applicant: NESTEC S.A., Vevey (CH)

(72) Inventors: Bing Wang, New South Wales (AU); Zhiqiang Zheng, Shanghai (CN); Junkuan Wang, Beijing (CN); Zhizhong Dong, Beijing (CN)

(73) Assignee: NESTEC S.A., Vevey (CH)

(21) Appl. No.: 14/916,125

(22) PCT Filed: Jun. 27, 2014
(86) PCT No.: PCT/EP2014/063742

§ 371 Date: Mar. 2, 2016

(30) Foreign Application Priority Data
Sep. 2, 2013 (CN) PCT/CN2013/082809

(51) Int. Cl. A61K 38/40

(52) U.S. Cl. CPC A61K 38/40; A23L 1/3056; A23L 1/296; A23V 2002/00

(57) ABSTRACT

The present invention generally relates to the development of cognitive function in infants. More particularly, the present invention provides the use of lactoferrin for improving memory and/or learning speed, and/or for promoting brain maturation in infants under physiological, i.e. non-pathological conditions. In one aspect, the present invention shows the utility of lactoferrin for improving long-term memory, e.g. long-term location memory in a healthy infant.

The present invention generally relates to the development of cognitive function in infants. More particularly, the present invention provides the use of lactoferrin for improving memory and/or learning speed, and/or for promoting brain maturation in infants under physiological, i.e. non-pathological conditions. In one aspect, the present invention shows the utility of lactoferrin for improving long-term memory, e.g. long-term location memory in a healthy infant.

### BACKGROUND OF THE INVENTION

Lactoferrin (LF) is a whey-fraction associated 80-kDa glycoprotein composed of 703-amino acid residues and one to four molecules of terminal sialic acid (Sia) residues on their N-linked oligosaccharide chains. Lactoferrin was originally isolated from milk, but is also found in other bodily fluids including tears, saliva, vaginal fluids, semen, nasal and bronchial secretions, bile, gastrointestinal fluids, urine, and is particularly abundant in human colostrum (6 g/l) and mature milk (2 g/l) [1-4]. It belongs to the transferrin family and is also known as lactotransferrin (LTF). Lactoferrin shows many biological functions for infants such as regulation of iron absorption in the bowel, immune response, antioxidant, anticarcinogenic, anti-inflammatory properties, and protection against microbial infection [5, 6].

Mother's milk is recommended for all infants. However, in some cases breast feeding is inadequate or unsuccessful or inadvisable for medical reasons, or the mother chooses not to breast feed either at all or for a period of more than a few weeks. Infant feeding formulas have been developed for these situations. Instant feeding formulas are commonly used today to provide supplemental or sole source nutrition early in life. They may be used instead of or in addition to mother's milk to feed infants. Consequently, they are often designed today to resemble mother's milk as closely as possible in terms of composition and function.

Recently, evidence has been accumulated that breastfeeding may provide long-term cognitive advantages. However, the underlying mechanisms to explain the relationship between breast feeding and cognitive development remains unclear. Lactoferrin is the second most abundant protein in human milk which is only less than caseins [7]. Interestingly, there are one to four sialic acid residues for each lactoferrin molecule, and animal experiments suggest that sialic acid may be involved in learning and memory [8, 9].

It was thus considered by the inventors that lactoferrin might have a role as a conditional nutrient for the infants' brain development and cognitive function when brain undergoes rapid growth. If so, early ingestion of lactoferrin should have a significant impact on brain structure and function from fetus to later life.

Cognition refers to information processing abilities, including perception, learning, memory, judgment and problem solving. The assessment of cognitive function is the central aspect of neuroscientific studies of the relationship between mechanism and functions. In general, learning and memory are considered to require higher brain functions, rather than the acquisition of simple neuron responses [10, 11].

In particular, memory is an organism's mental ability to store, retain and recall information. Memory phenomena that can be examined include: (1) knowledge (what to remember), (2) comprehension (what does it mean); (3) context/function (why to remember); and (4) strategy (how to remember). Memory is a complex psychological process that is not independent of a single memory domain process. Memory is related to several other cognition domains including, sensory memory, audio memory and visual memory.

Aspects of memory include:

Memory is a process in which information is encoded, stored, and retrieved. Encoding allows information that is from the outside world to reach an animal's senses in the form of chemical and physical stimuli. Storage is the second memory stage or process. This entails that an animal, such as a human, maintains information over periods of time. Finally, the third process is the retrieval of information that was stored. Such information must be localized and returned to the consciousness.

Short-term memory (STM) allows recalling something for a period of several seconds to a minute without rehearsal. Short-term memory encodes e.g. acoustical information, is supported by transient patterns of neuronal communication, and depends on regions of the frontal lobe (especially dorsolateral prefrontal cortex) and the parietal lobe, which stores items for only a few seconds.

Working memory overlaps with short-term memory to some extent. It is conceptualized as an active system for temporarily storing, processing and manipulating information needed in the execution of complex cognitive tasks (e.g., learning, reasoning, and comprehension).

Animal working memory means a short-term memory for an object, stimulus, or location that is used within a testing session, but not typically between sessions.

Long-term memory (LTM) is maintained by more stable and permanent changes in neural connections widely spread throughout the brain that can last as little as a few days or as long as decades. Long-term memory can store much larger quantities of information. Without the hippocampus, new memories are unable to be stored into long-term memory.

Spatial memory. In cognitive psychology and neuroscience, spatial memory is the part of memory responsible for recording information about an animal's environment and its spatial orientation. It is often argued that in both humans and other animals, spatial memories are summarized as a cognitive map. Spatial memory has representations within working, short-term and long-term memory. Research indicates that there are specific areas of the brain associated with spatial memory.

Location memory, also referred to as object-location memory, is an important form of spatial memory, comprising different subcomponents each of which processing specific types of information within memory, i.e. remembering objects, remembering positions, remembering the location of objects relative to each other, and binding these features in memory.

Learning is acquiring new, or modifying and reinforcing existing, knowledge, behaviors, skills, values, or preferences and may involve synthesizing different types of information.

When assessing the utility of an animal model for investigating cognitive function such as learning and memory, it is necessary to evaluate which species is most suitable. The potential for using pigs in pediatric brain research was recognized more than 40 years ago, due to the similarities in the whole brain growth at the time of birth, the gross anatomy, the growth pattern of neonatal brain to that of human. The pig digestive system shares similar physiology and anatomical structure with human infants and has comparable nutrient requirement. These make piglet ideally suitable for the coordinated nutritional, metabolic and molecular investigation [8]. The pig has the potential to fill the gap between preclinical studies with rodents and clinical trials in humans [11, 12].

Some studies addressed the potential benefit of lactoferrin as a dietary supplement:

WO 2010/130641 relates to neuronal health and development in the infant gut. Compositions comprising lactoferrin were shown to be useful in the promotion of the enteric nervous system, in the repair of an impaired enteric nervous system, and in treating or preventing disorders linked to a delayed development of the enteric nervous system.

WO 2010/130643 relates to brain health and development in infants. Compositions supplemented with lactoferrin were shown to be useful in the treatment or prevention of a delayed brain or nervous system development, in particular in IUGR (intrauterine growth restriction) infants (as shown in the model of dexamethasone induced preterm delivery).

WO 2010/130646 relates to brain health and brain protection in adults. Compositions supplemented with lactoferrin were shown to be useful in maintaining cognitive function and preventing cognitive decline and cognitive disorders.

WO 2013/076101 relates to the white matter. Compositions comprising lactoferrin were shown to be useful in the promotion of the development of the white matter, in the treatment or prevention of a delayed development of the white matter, and in the treatment or prevention of a loss of white matter.

US 2013/0150306 relates to milk-based nutritional compositions containing lactoferrin. Particularly disclosed is the administration of lactoferrin from a non-human source to a child with the purpose of modulating psychological stress.

None of these studies addressed the issue of memory and learning in healthy infants.

It was thus an object of the present invention to provide further beneficial uses of lactoferrin.

### SUMMARY OF THE INVENTION

The aim of the present invention is achieved by subject-matter as specified in the independent claims. Particular embodiments of the invention are as specified in the dependent claims.

The object of the present invention is solved by the use of lactoferrin for improving memory in a healthy infant.

In one embodiment, the memory is spatial memory, preferably location memory.

In one embodiment, the memory is long-term memory, preferably long-term spatial memory, more preferably long-term location memory.

The object of the present invention is further solved by the use of lactoferrin for improving learning speed in a healthy infant.

The object of the present invention is further solved by the use of lactoferrin for promoting brain maturation in a healthy infant.

In one embodiment, the lactoferrin is administered to the healthy infant at a daily intake dose in the range of 100 to 400 mg/kg body wt/day or 105 to 350 mg/kg body wt/day or 125 to 350 mg/kg body wt/day or 110 to 300 mg/kg body wt/day, preferably 140 to 290 mg/kg body wt/day or 120 to 270 mg/kg body wt/day or 145 to 285 mg/kg body wt/day.

In a preferred embodiment, the daily intake dose of lactoferrin is a medium dose, e.g. in the range of 100 to 200 mg/kg body wt/day or 100 to 175 mg/kg body wt/day or 110 to 160 mg/kg body wt/day or 120 to 150 mg/kg body wt/day, preferably 128 mg/kg body wt/day or 145 mg/kg body wt/day. Herein, a “medium dose” may also be referred to as “intermediate dose” or “sufficient dose”.

In a particularly preferred embodiment, the lactoferrin is administered to the healthy infant at a medium daily intake dose (e.g. as specified in the preceding paragraph) for improving learning speed and/or long-term memory.

In another preferred embodiment, the daily intake dose of lactoferrin is a high dose, e.g. in the range of 220 to 320 mg/kg body wt/day or 225 to 325 mg/kg body wt/day or 250 to 350 mg/kg body wt/day or 230 to 310 mg/kg body wt/day or 240 to 300 mg/kg body wt/day, preferably 252 mg/kg body wt/day or 285 mg/kg body wt/day.

In a particular preferred embodiment, the lactoferrin is administered to the healthy infant at a high daily intake dose (e.g. as specified in the preceding paragraph) for improving long-term memory, in particular long-term location memory.

In one embodiment, the daily intake dose of lactoferrin is split up into at least two, preferably at least three, most preferably four portions.

In one embodiment, the lactoferrin is provided in an ingestible composition, preferably a liquid ingestible composition, selected from the group consisting of human food products, maternal nutritional compositions, starter milks, growing up milks, infant feeding formulas and baby food and drinks.

In one embodiment the lactoferrin is present in a liquid ingestible composition at a concentration in the range of 0.1 to 2 g/l, preferably 0.25 to 1.5 g/l, most preferably 0.5 to 1.0 g/l.

In a preferred embodiment, the lactoferrin is present in the liquid ingestible composition at a concentration in the range of 0.3 to 0.7 g/l, preferably at a concentration of 0.5 g/l. Preferably this liquid ingestible composition is administered to the healthy infant for improving learning speed and/or long-term memory.

In an alternative preferred embodiment, the lactoferrin is present in the liquid ingestible composition at a concentration in the range of 0.8 to 1.2 g/l, preferably at a concentration of 1.0 g/l. Preferably this liquid ingestible composition is administered to the healthy infant for improving long-term memory, in particular long-term location memory.

In one embodiment, the lactoferrin is provided to the healthy infant as a milk or whey fraction enriched in lactoferrin.

In the above described uses of lactoferrin for improving memory in a healthy infant, lactoferrin may be used in an ingestible composition enriched in lactoferrin. Enriched means that lactoferrin was either added to the composition, so that the resulting lactoferrin content of the composition is higher than the lactoferrin content of the composition without lactoferrin addition, or that the composition was treated in a way to concentrate the natural lactoferrin content in a composition.

Lactoferrin may also be provided as pure compound.

Alternatively, lactoferrin may be provided as a lactoferrin enriched fraction, for example a lactoferrin enriched milk or whey fraction.

As milk or whey source bovine milk, human milk, goat milk, camel milk, horse milk and/or donkey milk may be used, for example. Colostrum may be used as well.

Compositions are administered in an amount sufficient to be effective. An amount adequate to accomplish this is defined as “an effective dose”. Amounts effective will depend on a number of factors known to those of skill in the art. The precise amounts depend on a number of individual factors such as the infant's development stage or weight.

Typical lactoferrin enriched compositions may comprise lactoferrin in an amount of at least 1.6 g/l.

For example, a composition used in the present invention may contain lactoferrin in a concentration of at least 0.75% (w/w), preferably at least 1% (w/w). In one embodiment, the composition is to be administered in an amount corresponding to an ingestion of at least 0.25 g lactoferrin, preferably at least 0.5 g lactoferrin more preferably at least 1 g lactoferrin per day per kg body weight.

Lactoferrin may be present in the composition in a concentration of at least 0.01 g per 100 kcal, preferably of at least 0.1 g per 100 kcal. For example, lactoferrin may be present in the composition in the range of about 0.01 g to 100 g, preferably 0.1 g to 50 g, even more preferred 2 g to 25 g per 100 kcal of the composition.

Lactoferrin may also be used in combination with other compounds, such as sialic acid and/or iron, for example.

A particular preferred lactoferrin containing composition may contain additionally sialic acid in an amount in the range of 100 mg/100 g (w/w) to 1000 mg/100 g (w/w) of the composition, for example in the range of 500 mg/100 g (w/w) to 650 mg/100 g (w/w) of the composition.

The composition used in the present invention may for example comprise at least about 0.001% sialic acid (by weight). In further embodiments of the present invention, the composition may comprise at least about 0.005% or at least about 0.01% of sialic acid (by weight)

Alternatively or additionally the lactoferrin containing composition may contain iron in an amount in the range of about 1 mg/100 g (w/w) to 50 mg/100 g (w/w) of the composition, for example 10 mg/100 g (w/w) to 30 mg/100 g (w/w) of the composition.

One lactoferrin containing composition may contain for example about 852 mg/100 g (w/w) sialic acid and 22 mg/100 g (w/w) iron.

The lactoferrin containing composition of the present invention may have a caloric density in the range of 30 kcal/100 g-1000 kcal/100 g of the composition, preferably 50 kcal/100 g-450 kcal/100 g of the composition. It may for example have a caloric density of about 400 kcal/100 g.

The nature of the composition is not particularly limited. It is preferably a composition for oral or enteral administration.

The composition may be for example selected from the group consisting of food products, animal food products, pharmaceutical compositions, nutritional formulations, nutraceuticals, drinks, food additives, and infant feeding formulas.

In one typical embodiment, the composition will contain a protein source, a lipid source and a carbohydrate source.

For example such a composition may comprise protein in the range of about 2 to 6 g/100 kcal, lipids in the range of about 1.5 to 3 g/100 kcal and/or carbohydrates in the range of about 1.7 to 12 g/100 kcal If the composition is liquid, its energy density may be between 60 and 75 kcal/100 ml.

If the composition is solid, its energy density may be between 60 and 75 kcal/100 g.

The type of protein is not believed to be critical to the present invention. Thus, protein sources based on whey, casein and mixtures thereof may be used, for example. As far as whey proteins are concerned, acid whey or sweet whey or mixtures thereof may be used as well as alpha-lactalbumin and beta-lactoglobulin in whatever proportions are desired. The whey protein may be modified sweet whey. Sweet whey is a readily available by-product of cheese making and is frequently used in the manufacture of infant formulas based on cows' milk. However, sweet whey includes a component which is undesirably rich in threonine and poor in tryptophan called caseino-glyco-macropeptide (CGMP). Removal of the CGMP from sweet whey results in a protein with a threonine content closer to that of human milk. This modified sweet whey may then be supplemented with those amino acids in respect of which it has a low content (principally histidine and tryptophan). A process for removing CGMP from sweet whey is described in EP 880902 and an infant formula based on this modified sweet whey is described in WO 01/11990. The proteins may be intact or hydrolyzed or a mixture of intact and hydrolyzed proteins. It may be desirable to supply partially hydrolysed proteins (degree of hydrolysis between 2 and 20%), for example for subjects believed to be at risk of developing cow's milk allergy. If hydrolysed proteins are required, the hydrolysis process may be carried out as desired and as is known in the art. For example, a whey protein hydrolysate may be prepared by enzymatically hydrolysing the whey fraction in two steps as described in EP 322589. For an extensively hydrolysed protein, the whey proteins may be subjected to triple hydrolysis using Alcalase 2.4L (EC 940459), then Neutrase 0.5L (obtainable from Novo Nordisk Ferment AG) and then pancreatin at 55 [deg.]C. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

The compositions used in the present invention may contain a carbohydrate source. Any carbohydrate source may be used, such as lactose, saccharose, maltodextrin, starch and mixtures thereof.

The compositions used in present invention may contain a lipid source. The lipid source may be any lipid. Preferred fat sources include milk fat, palm olein, high oleic sunflower oil and high oleic safflower oil. The essential fatty acids linoleic and [alpha]-linolenic acid may also be added as may small amounts of oils containing high quantities of preformed arachidonic acid and docosahexaenoic acid such as fish oils or microbial oils. The lipid source preferably has a ratio of n-6 to n-3 fatty acids of about 5:1 to about 15:1; for example about 8:1 to about 10:1.

The compositions of the present invention may also contain all vitamins and minerals understood to be essential in the daily diet and in nutritionally significant amounts. Minimum requirements have been established for certain vitamins and minerals. Examples of minerals, vitamins and other nutrients optionally present in the infant formula include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. Minerals are usually added in salt form. The presence and amounts of specific minerals and other vitamins will vary depending on the numerous factors, such as age weight and condition of the person or animal the composition is administered to.

The compositions may also comprise at least one probiotic bacterial strain. A probiotic is a microbial cell preparation or components of microbial cells with a beneficial effect on the health or well-being of the host. The amount of probiotic, if present, likewise preferably varies as a function of the age of the person or animal. Generally speaking, the probiotic content may increase with increasing age of the infant for example from 10<3> to 10<12> cfu/g formula, more preferably between 10<4> and 10<8> cfu/g formula (dry weight).

The compositions may also contain at least one prebiotic in an amount of 0.3 to 10%. A prebiotic is a non-digestible food ingredient that beneficially affects the host by selectively stimulating the growth and/or activity of one or a limited number of bacteria in the colon, and thus improves host health. Such ingredients are non-digestible in the sense that they are not broken down and absorbed in the stomach or small intestine and thus pass intact to the colon where they are selectively fermented by the beneficial bacteria. Examples of prebiotics include certain oligosaccharides, such as fructo-oligosaccharides (FOS) and galacto-oligosaccharides (GOS). A combination of prebiotics may be used such as 90% GOS with 10% short chain fructo-oligosaccharides such as the product sold under the trade mark Raftilose® or 10% inulin such as the product sold under the trade mark Raftiline®.

The compositions may optionally contain other substances which may have a beneficial effect such as nucleotides, nucleosides, and the like.

The compositions, for example an infant formula, for use in the invention may be prepared in any suitable manner. For example, an infant formula may be prepared by blending together the protein source, the carbohydrate source, and the fat source in appropriate proportions. If used, the emulsifiers may be included in the blend. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The liquid mixture may then be thermally treated to reduce bacterial loads. For example, the liquid mixture may be rapidly heated to a temperature in the range of about 80<0>C to about 110<0>C for about 5 seconds to about 5 minutes. This may be carried out by steam injection or by heat exchanger; for example a plate heat exchanger. The liquid mixture may then be cooled to about 60<0>C to about 85 [deg.]C; for example by flash cooling. The liquid mixture may then be homogenised; for example in two stages at about 7 MPa to about 40 MPa in the first stage and about 2 MPa to about 14 MPa in the second stage. The homogenised mixture may then be further cooled to add any heat sensitive components; such as vitamins and minerals. The pH and solids content of the homogenised mixture is conveniently standardised at this point. The homogenised mixture is transferred to a suitable drying apparatus such as a spray drier or freeze drier and converted to powder. The powder should have a moisture content of less than about 5% by weight. If it is desired to add probiotic(s), they may be cultured according to any suitable method and prepared for addition to the infant formula by freeze-drying or spray-drying for example. Alternatively, bacterial preparations can be bought from specialist suppliers such as Christian Hansen and Morinaga already prepared in a suitable form for addition to food products such as infant formula. Such bacterial preparations may be added to the powdered infant formula by dry mixing.

Lactoferrin may be added at any stage during this procedure, but is preferably added after a heating step.

The composition comprises a protein source which may be present in the range of between 1.4 and 100 g/100 kcal, preferably between 1.4 and 6.0 g/100 kcal of the composition. Since lactoferrin is a protein it should be considered a part of the protein source.

Whey protein is known to provide several health benefits. For example, it is easily digestible. The protein fraction in whey (approximately 10% of the total dry solids within whey) comprises several protein fractions, for example beta-lactoglobulin, alpha-lactalbumin, bovine serum albumin and immunoglobulins. In one embodiment at least 50%, preferably at least 75%, even more preferred at least 85% by weight of the protein source is whey protein.

If present, the lipid source may contribute to between 30 to 55% of the total energy of the composition. A carbohydrate source may contribute to between 35 and 65% of the total energy of the composition.

Sialic acid may also be added to the composition of the present invention. Sialic acid is a generic term for the N- or O-substituted derivatives of neuraminic acid, a monosaccharide with a nine-carbon backbone.

### DETAILED DESCRIPTION OF THE INVENTION

The present invention is based on the finding that dietary supplementation of lactoferrin improves location memory, learning speed and long-term memory in piglets under physiological conditions.

In a first set of experiments, lactoferrin was shown to improve location memory of 36- to 38-day-old piglets. In particularly, the 30-min long-term location memory of piglets fed with high doses of lactoferrin (about 225 to 325 mg/kg body wt/day) turned out to be significantly better than that of piglets in the control group.

In a second set of experiments the data indicated that dietary lactoferrin supplementation strongly promotes memory and learning speed as tested on 22- to 32-day-old piglets in the 8-arm radial maze. The 40-min and 3-h long-term memory turned out to be improved in piglets fed with both medium doses (about 100 to 175 mg/kg body wt/day) and high doses of lactoferrin. Interestingly, learning speed was enhanced best with medium doses of lactoferrin.

In another set of experiments it was shown that lactoferrin supplementation up-regulated Brain Derived Neurotrophin Factor (BDNF) mRNA expression in the hippocampus, and that it activated the BDNF signaling pathway.

The activation of the BDNF signaling pathway could be seen by the up-regulation of key genes in this pathway e.g. Trk3, IRS1, GRB2, CAMK1, MAPK, SP1 and CREB1.

It was also shown that lactoferrin supplementation increased the level of phosphorylated phosphorylate CREB (pCREB) in the hippocampus.

It is well established that activation of the BDNF signaling pathway leads to enhances phosphorolation and nuclear translocation of CREB. It is also established the phosphorylation of CREB at serine 133 (pCREB) induces gene transcription, and plays a critical role in initiating learning and memory processes.

In another set of experiments the data indicated that dietary lactoferrin supplementation led to a higher level of polySialic acid-Neural Cell Adhesion Molecule (NCAM) expression in the hippocampus and pre-frontal cortex. This indicates that the sialic acid moiety of lactoferrin may be a key factor in increasing neuroplasticity and facilitating LTM consolidation.

Lactoferrin as used according to the present invention may be obtained from various sources. It may be purified, e.g. from milk or whey, or may be produced recombinantly. Lactoferrin purified from a natural source has the advantage that it is a natural ingredient, mostly obtained from a food-grade composition, and can be used as enriched fraction of a food composition with or without further purification. As a natural source, human milk (mother's milk) or milk from a non-human source, e.g. cow's milk, goat's milk, camel's milk, horse's milk or donkey's milk, is considered. Further considered is colostrum. Recombinantly obtained lactoferrin has the advantage that it can be produced easily in high concentrations.

The lactoferrin may be added to a composition, so that the resulting lactoferrin content of the composition is higher than the lactoferrin content of the composition without lactoferrin addition. Alternatively, a composition naturally containing lactoferrin may be treated in order to concentrate the natural lactoferrin content in the composition resulting in, e.g. a lactoferrin enriched milk or whey fraction. Lactoferrin may also be provided as pure compound.

The lactoferrin may be provided as an ingredient of an ingestible composition, i.e. a composition for oral administration. Compositions for enteral administration are also considered.

The lactoferrin, or an ingestible composition comprising lactoferrin, preferably is administered to the infant. Administration to the mother during the gestation and/or lactation period is also considered.

The lactoferrin may also be provided in combination with other compounds, such as sialic acid and/or iron.

The term “memory” and its various aspects are used in the claims within the meaning as e.g. outlined in the introductory section.

“Learning speed” refers to the duration of time or the number of trials needed to learn a learning task.

“Brain maturation” is mainly the process of brain development, including generating, shaping, and reshaping the nervous system, from the earliest stages of embryogenesis to the final years of life.

A “healthy infant” means an infant who was a full-term newborn (after 37 weeks of pregnancy, in case of a human newborn), i.e. a newborn delivered at term in contrast to preterm delivery, was of normal weight at birth, did not show any cognitive dysfunction or brain retardation at birth and did not experience intrauterine growth restriction (IUGR) or hypoxemia-ischemia at birth. Thus, in a broader sense, “healthy” relates to a normal or physiological, i.e. non-pathological development of an infant, in particular with regard to cognitive function.

The infant can be a newborn, a baby, a toddler, a pre-school child or school child, from birth up to the age of 14 years old.

A newborn is generally defined as a human from about birth to 1 month of age. A baby usually means a human at the age of 6 to 12 months old. A toddler is a human from 12 to 36 months. A pre-school child is a human from the age of 36 months to 5 years old. A school child is from 5 to 14 years of age.

Although a human healthy infant is preferred, non-human mammals of respective age are also considered.

Further advantages and features of the present invention will be apparent to those of skill in the art from the following examples and figures.

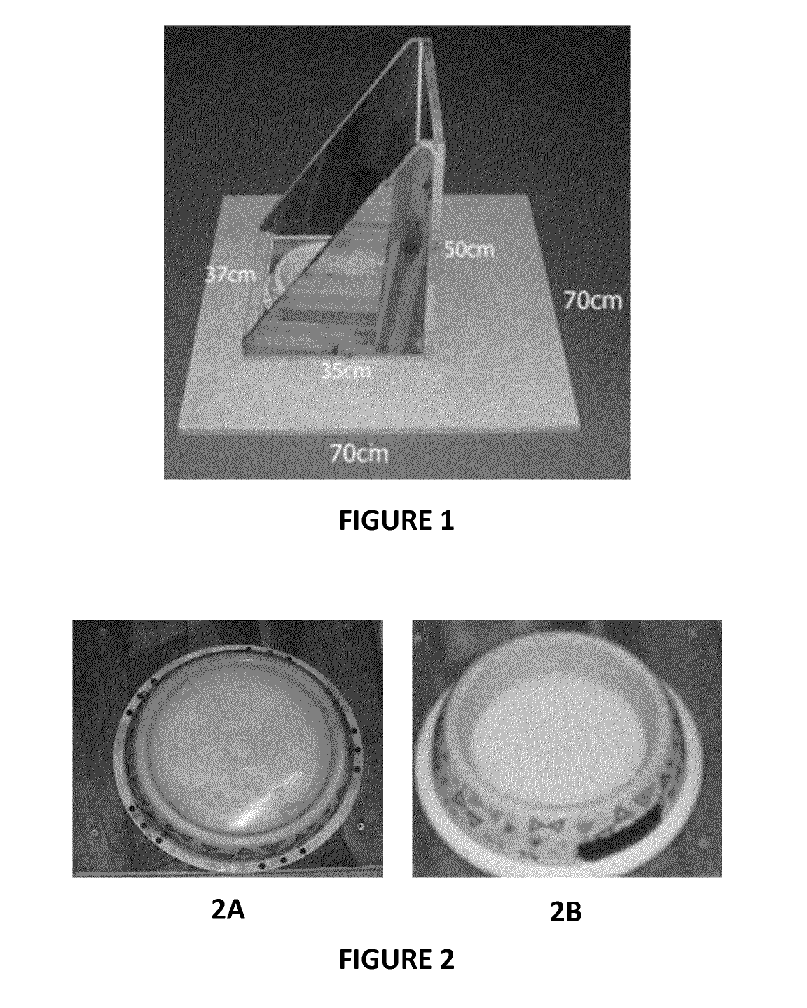

FIG. 1 shows one of four partition boards harboring a milk containing bowl.

FIG. 2 shows the situation when milk is not accessible to the piglet, i.e. a milk containing bowl is covered by a lid (FIG. 2A), and, in contrast, when milk is accessible, i.e. the bowl is uncovered (FIG. 2B).

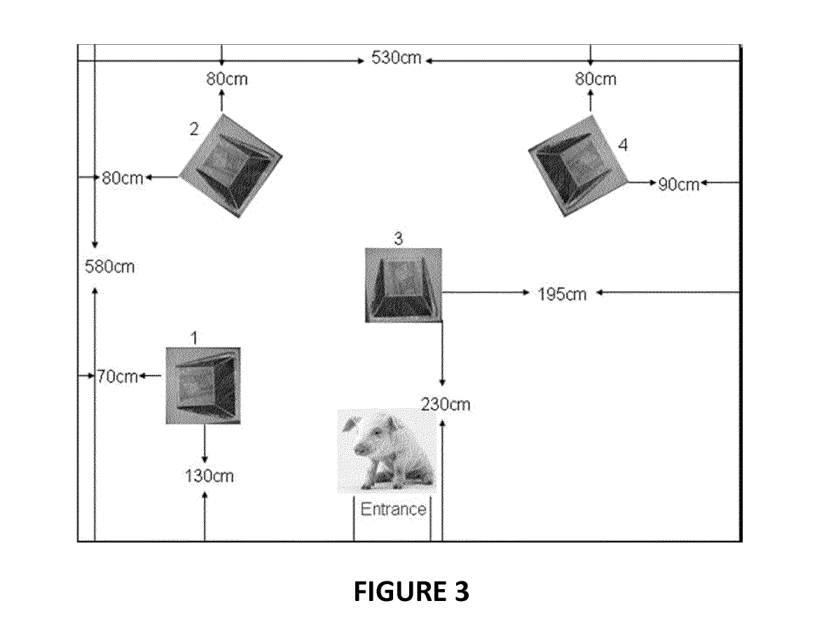

FIG. 3 illustrates the location memory test arrangement.

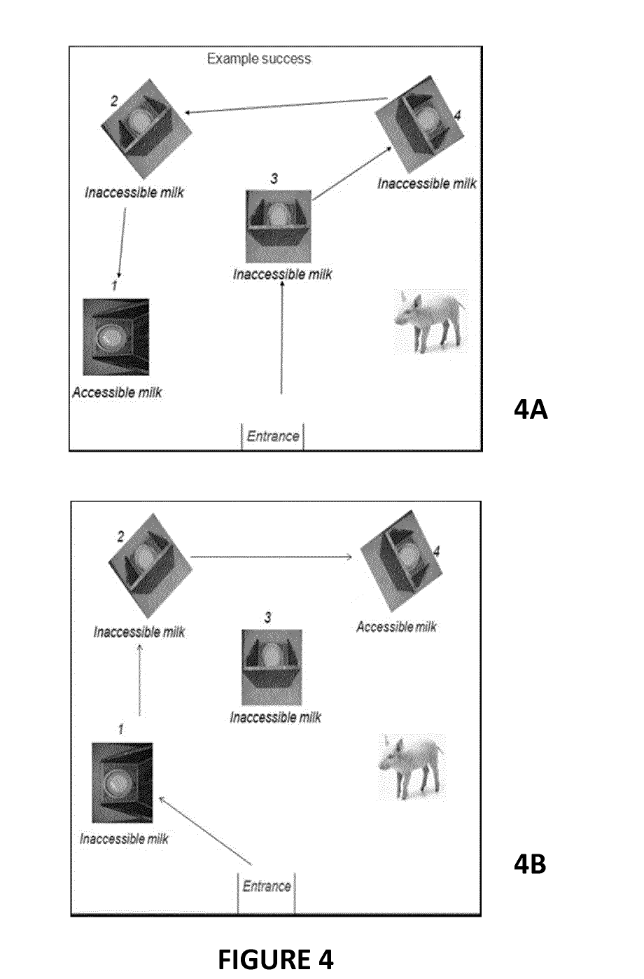

FIG. 4 illustrates the location memory test arrangement in case of mistake (FIG. 4A) or success (FIG. 4B). An indication of success is when the piglet first visits the bowl in which milk was accessible in the previous run.

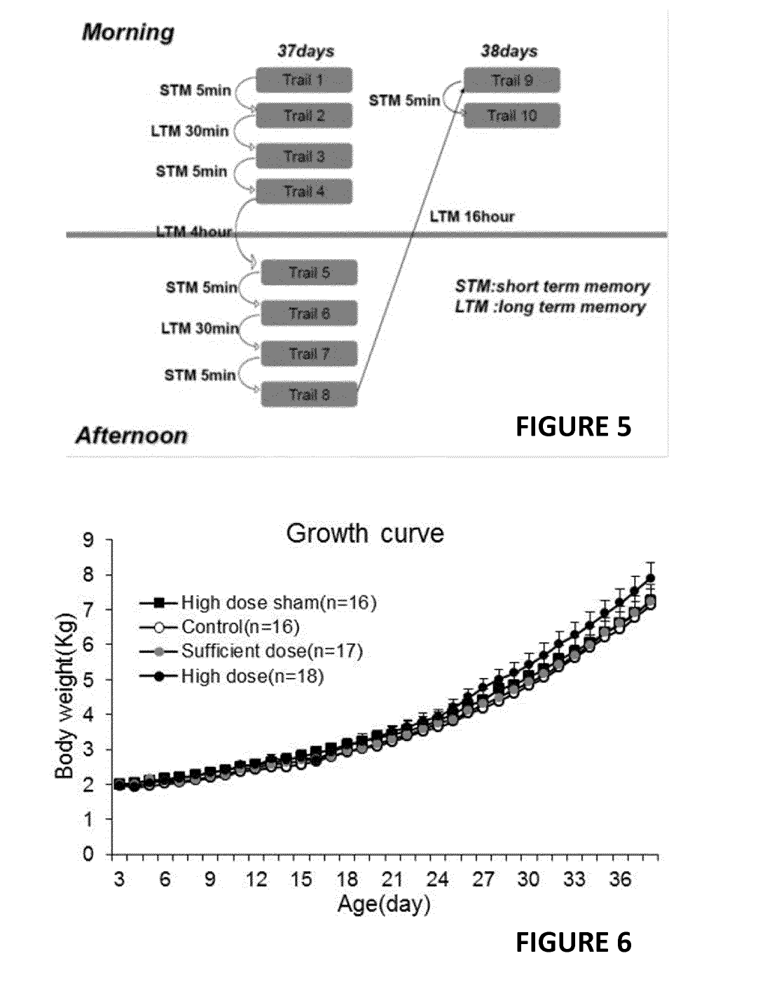

FIG. 5 shows the time schedule used in the location memory test arrangement.

FIG. 6 shows the mean (±SE) weight gain in each group throughout the study. There were not significant differences among the groups (P>0.05) based on a general linear model (univariate ANOVA) with Bonferroni's adjustment for multiple comparisons.

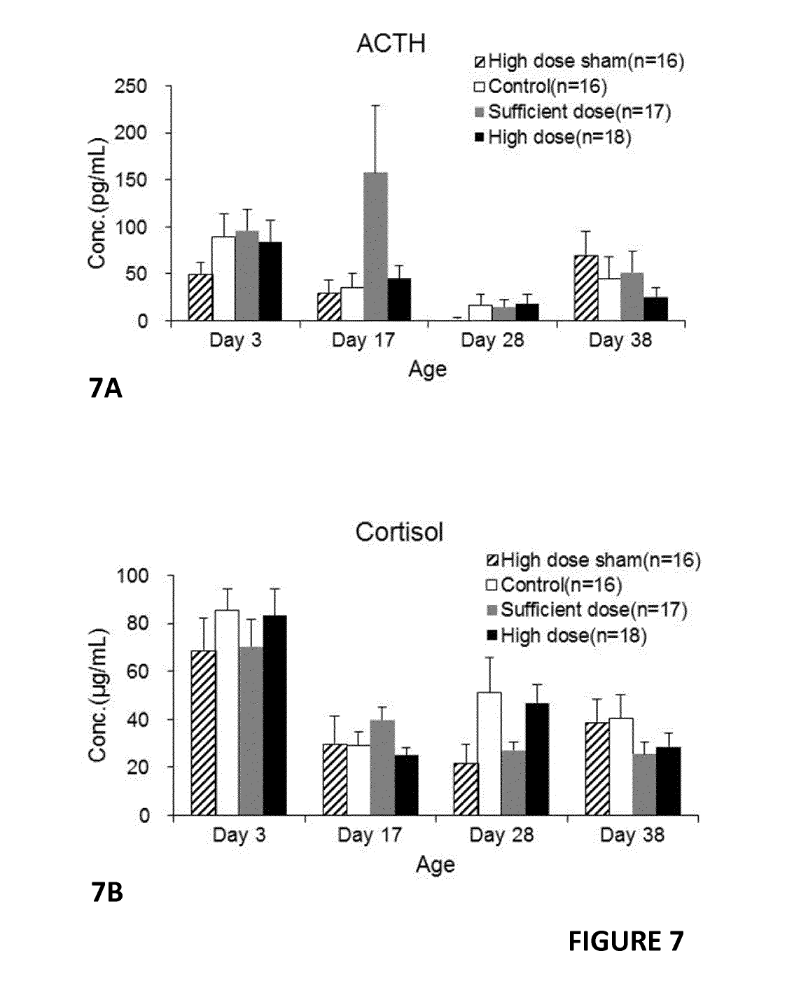

FIG. 7 shows the blood concentration of stress hormones adrenocorticotropic hormone (ACTH) (FIG. 7A) or cortisol (FIG. 7B) at four different ages of the piglets. The concentration of ACTH is given as [pg/ml], that of cortisol as [μg/ml].

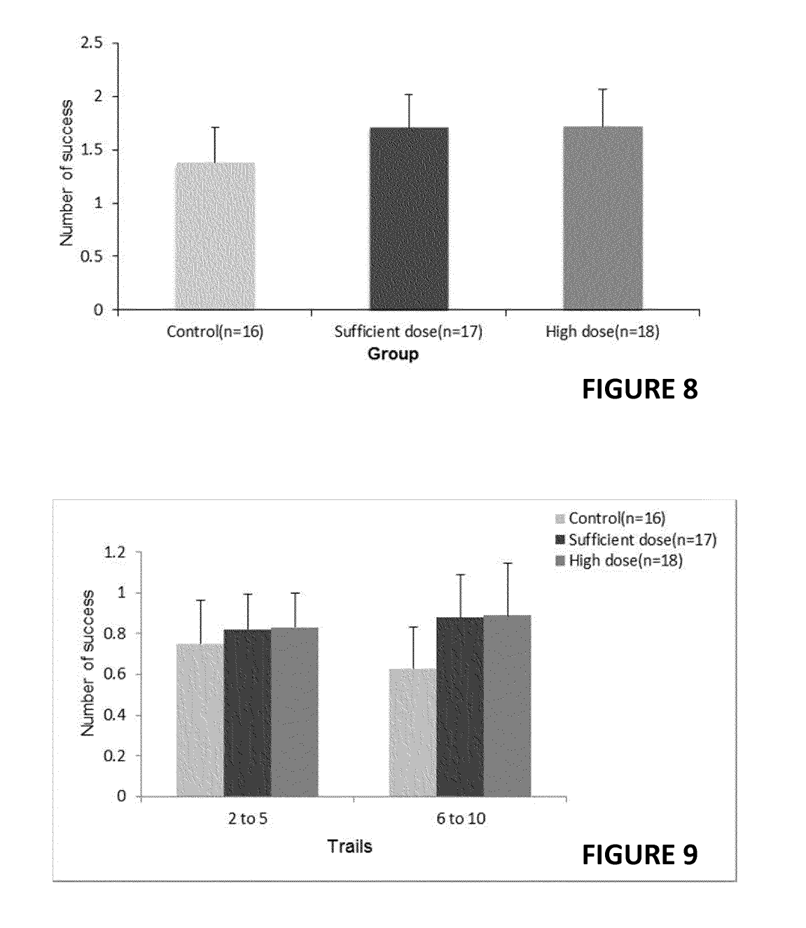

FIG. 8 shows the total number of successes with regard to location memory (10 trials).

FIG. 9 shows the number of successes with regard to location memory at different test stages.

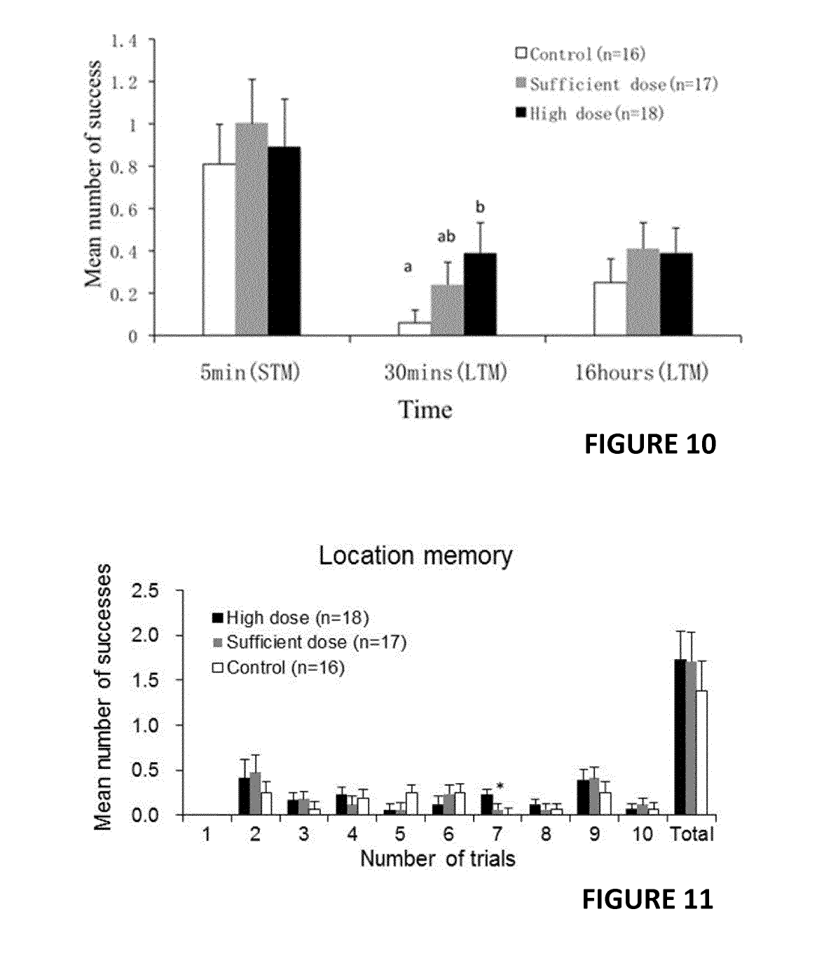

FIG. 10 shows the short-term (STM) and long-term (LTM) location memory (mean±SE). Different letters (a, ab, b) marking the bars relating to the 30 min LTM shows statistically significance between the groups (P=0.046) using one way ANOVA with LSD adjustment for multiple comparisons.

FIG. 11 shows the working memory at different time intervals (*P=0.026, <0.05; two-way ANOVA).

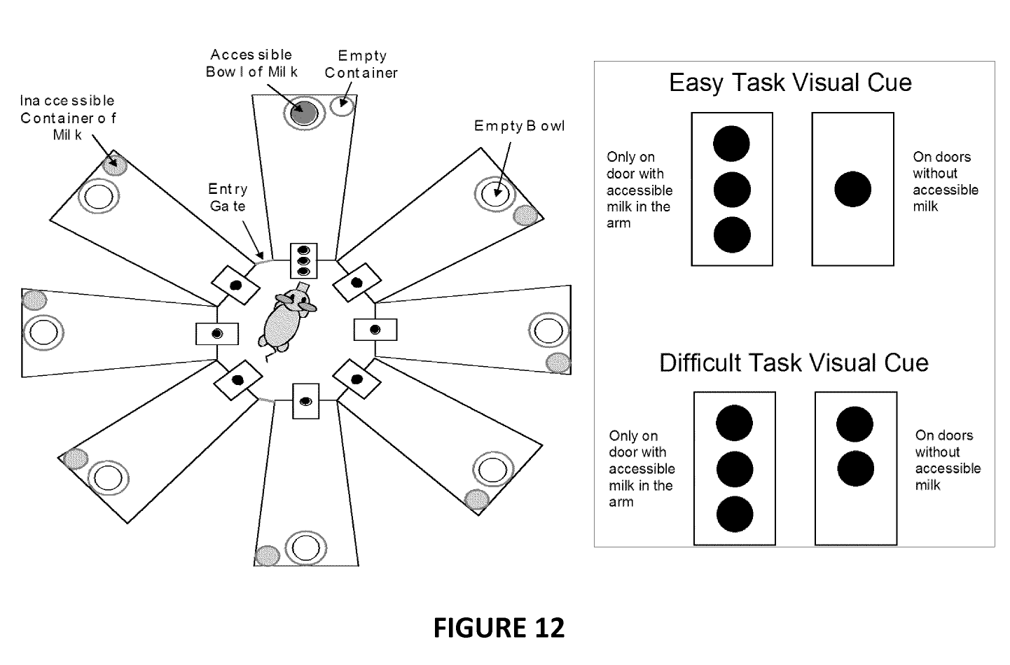

FIG. 12 shows an 8-arm radial maze and visual cure for easy task and difficult task.

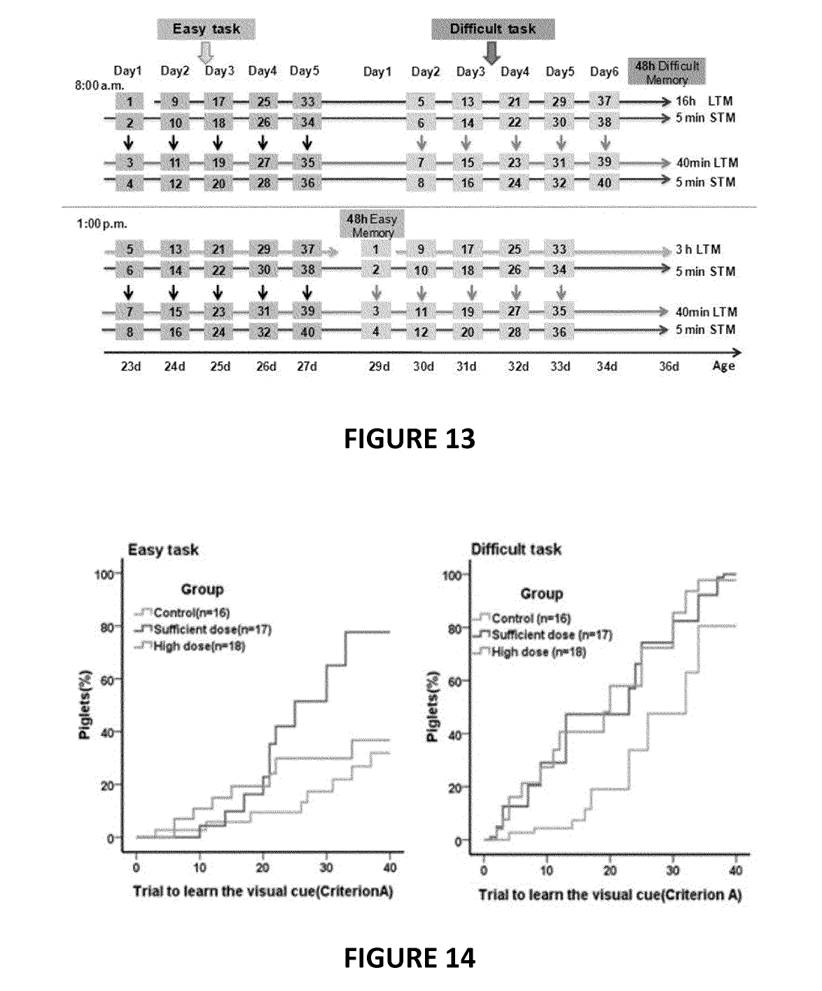

FIG. 13 shows the procedure of easy and difficult task at 8-arm radial maze. LTM: long-term memory, STM: short-term memory.

FIG. 14 shows the learning speed curve of piglets using mistake and success as a covariance analyzed using the Cox-regression method in easy and difficult tasks respectively.

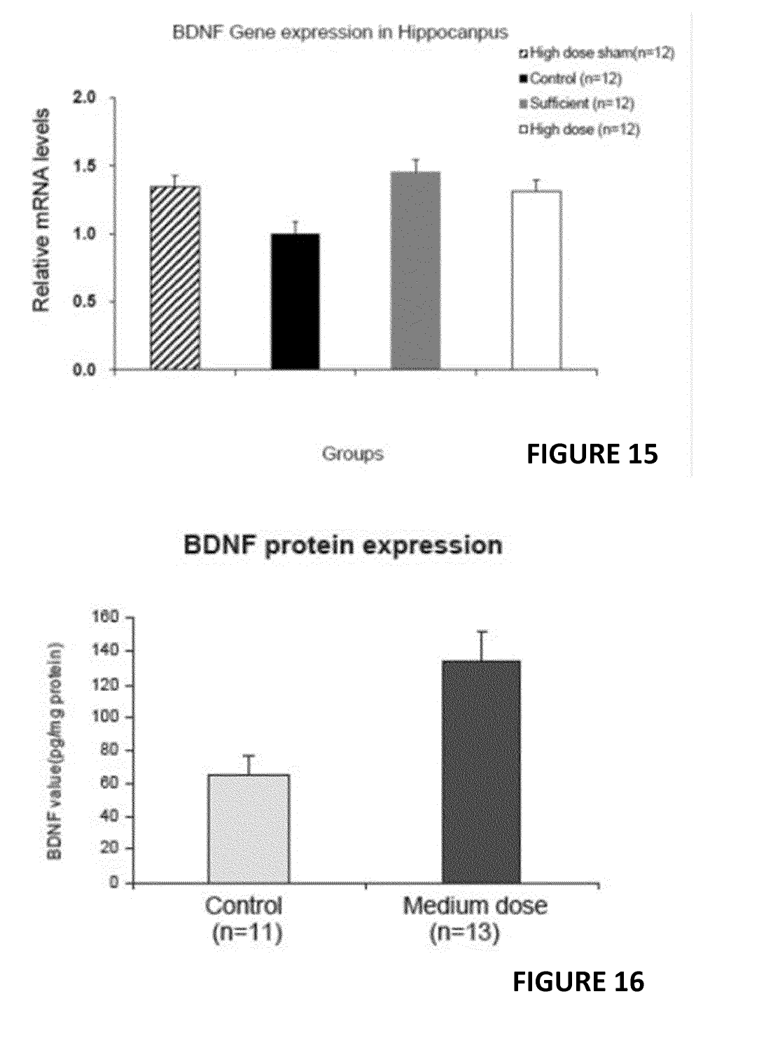

FIG. 15 shows the relative mRNA levels of brain-derived neutrotrophic factor (BDNF) in hippocampus (mean±SE). Lactoferrin supplementation significantly increases BDNF expression levels in hippocampus (P<0.05).

FIG. 16 shows the protein levels of BDNF in hippocampus (mean±SE). Medium dose of lactoferrin supplementation significantly increases BDNF protein levels in hippocampus (P<0.05).

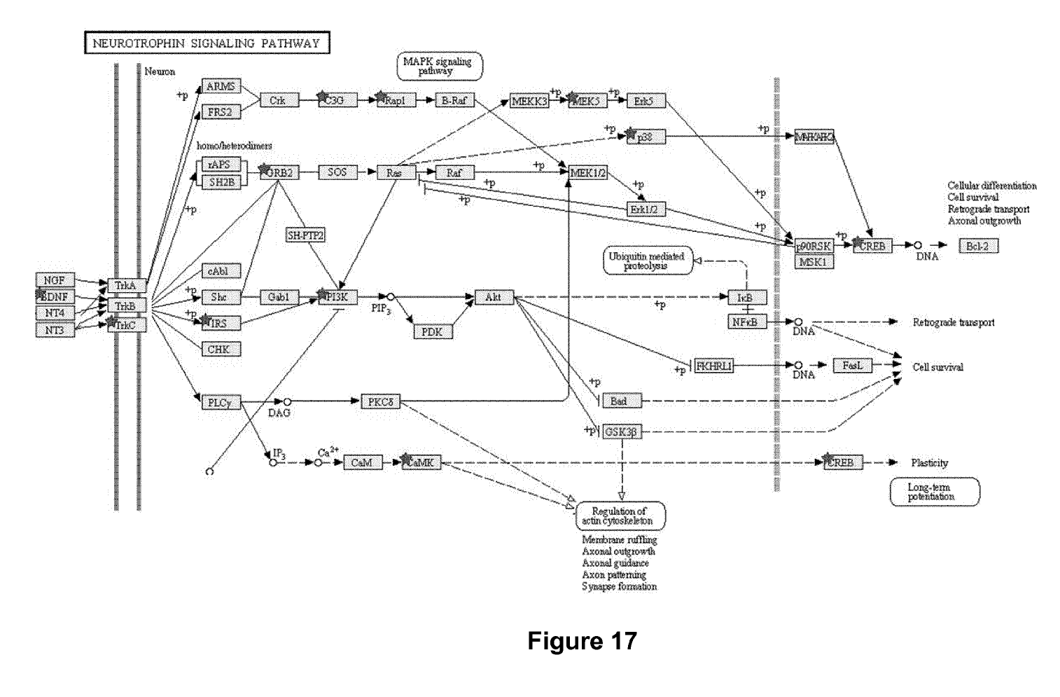

FIG. 17 shows the BDNF signaling pathway.

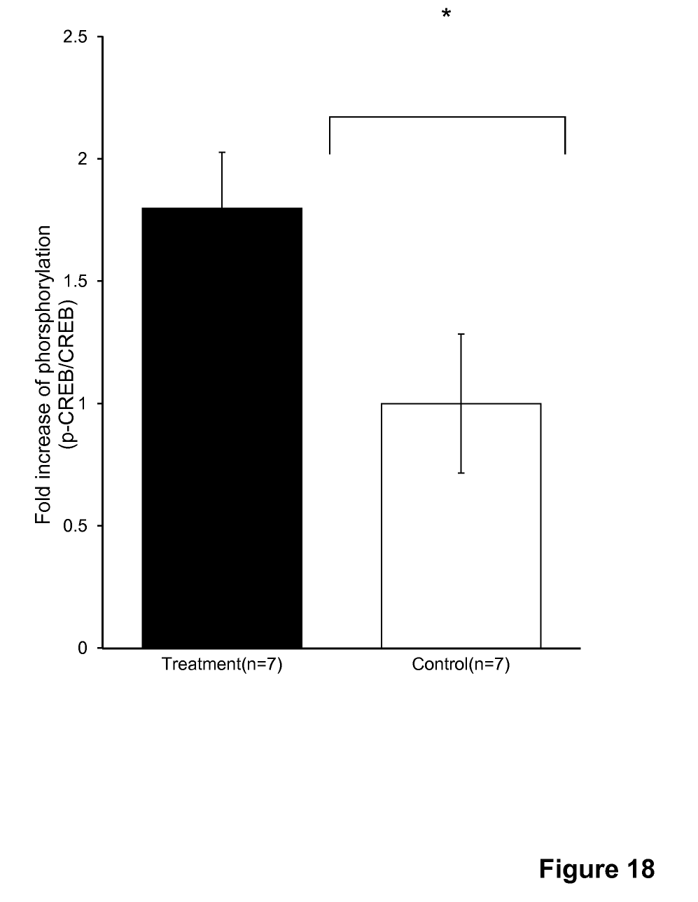

FIG. 18 shows that lactoferrin supplementation increased the level of pCREB in the hippocampus (mean±SE).

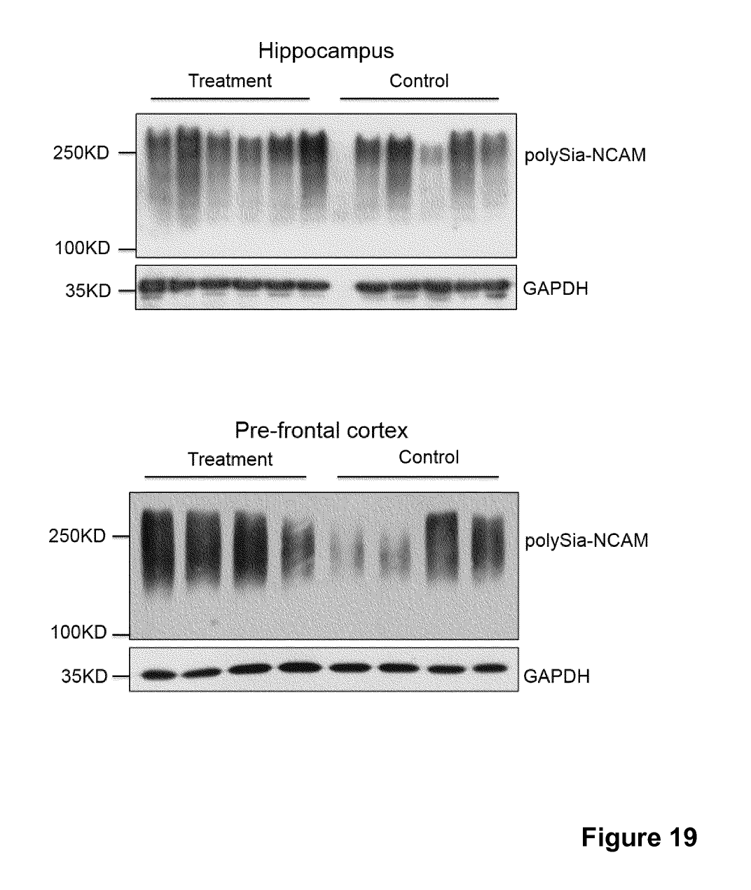

FIG. 19 shows that lactoferrin supplementation increased the level of polySia-NCAM in the hippocampus and pre-frontal cortext (mean±SE).

### EXAMPLES

### Example 1

### Animal Treatment

1.1. Animals

Piglets were used as an animal model because of the high similarity to human infants with regard to physiology, anatomy and genetics.

Sixty-seven 3-day-old male domestic piglets (Sus scorfa Landrace×Large White F1) from 16 litters were purchased and randomly assigned to 4 groups according to weight and litter. Grouping and diet information is outlined in Table 1. All animals were housed in pairs in a temperature controlled environment with a 12-h light (08:00-20:00) and dark (20:00-08:00) cycle. Each home pen contained a “nest” (a rubber tire covered with a clean towel), a heat lamp over the nest and an identical wooden toy. The maximum capacity of piglets at the behavior lab was 16. Thus, 5 trials of 10-16 piglets/trial were carried out to reach 16 piglets/group. The piglets were monitored with a camera surveillance system. Two 3-day-old piglets in each trial were euthanized and used as the baseline control (n=10). The study protocol was approved by the Xiamen University Animal Ethics Committee.

TABLE 1
Details of animal grouping

| | Group | | | |
|---|---|---|---|---|
| | Group 4 | Group 3 | Group 2 | Group 1 |
| | “sham” | “high dose” | “medium dose” | “control” |
| Behavioral test | No | Yes | Yes | Yes |
| Dose of lactoferrin | 1 g/l | 1 g/l | 0.5 g/l | 0.05 g/l |
| Total piglets/group | 16 | 18 | 17 | 16 |

1.2. Lactoferrin Feeding Protocol

Bovine milk lactoferrin (beta-lactoferrin) was purchased from DMV International. Each piglet (3-day-old to 38-day-old) was fed with a standard sow milk-replacer containing protein of soy/whey/casein (50:38:12). The amount of lactoferrin in the final milk varied depending on the group (see Table 1): 0.05 g/l (group 1, control group with no added lactoferrin, n=16), 0.5 g/l (group 2, medium dose, n=17), 1 g/l (group 3, high dose, n=15). All piglets in groups 1 to 3 were exposed to learning challenges. Group 4 (n=16) received lactoferrin at the same dose as group 3 (1 g/l), but was not exposed to learning challenges (served as sham group). These concentrations represented an approximate intake of lactoferrin in the control, medium dose and high dose group of 15, 145 and 285 mg/kg body wt/day, respectively. The pig milk replacers were formulated such that total protein intake remained the same irrespective of the amount of added lactoferrin. To maintain normal rates of growth, the piglets received 285 ml milk/kg body wt/day in the first 2 weeks of the study and 230 ml/kg body wt/day in the remaining weeks. Feeding times were at 08:00, 13:00, 18:00, and 22:30, with an extra 50 ml milk/pig supplied at the last feeding. Body weight, milk intake, and health status of piglets were recorded daily.

1.3. Body Weight

The piglets' body weight was measured every morning before feeding. The results showed that mean (±SE) starting body weight was the same in each group (1.908±0.044 kg), and animals gained weight at similar rates (FIG. 6). Although group 3 had a faster body weight gain than the other groups by the end of the study, differences between groups were not significant on day 23 (P=0.937), day 29 (P=0.899) and day 36 (P=0.888). Our results differ from a previous report according to which infants fed with a formula of lactoferrin and iron supplementation had a higher body weight gain than infants fed with a formula of iron supplementation alone [13].

1.4. Stress Hormones

It is well known that stressful experiences may affect learning and memory processes. Stress is generally defined as any condition that disturbs the physiological or psychological homeostasis of an organism [14]. Evidence from many different types of experiments indicates that adrenal stress hormones, released during or after emotionally arousing experiences, play a critical role in consolidating lasting memories. Stressful events activate the hypothalamus-pituitary-adrenal (HPA) axis, resulting in a slow increase in plasma corticosterone or cortisol levels [15]. Large amount of experiments investigating the effects of adrenal stress hormones on memory provide extensive evidence that epinephrine and glucocorticoids modulate long-term memory consolidation in animals and human subjects [16]. On that background, we monitored stress hormone levels in the piglets' blood.

Blood samples were collected from each piglet at the age of 3, 17, 28, and 39 days. The sample was centrifuged at 3000 rpm, 15 min at 4° C., and then blood plasma was stored at −80° C. until analysis. Two stress hormones, adrenocorticotropic hormone (ACTH) and cortisol, were measured in blood plasma by an electrochemiluminescence immunoassay on the Roche electrochemical luminescence immune analyzer (Roche E601, Zhongshan Hospital). All assays were handled according to manufacturer's instructions in all respects by experienced technicians. Quality control was performed for all analytical runs using control materials provided by the respective manufacturers. As a result, no effect on the stress hormone level in blood was found (FIG. 7; P>0.05, two-way ANOVA).

1.5. Statistical Analysis

Differences in memory between groups were carried out using a general linear model (univariate ANOVA) with Bonferroni's adjustment for multiple comparisons, if necessary. All statistical analyses were completed with the use of SPSS for WINDOWS 19 (SPSS Inc, Chicago, Ill.). A significance level of 0.05 was used.

### Example 2

### Effect of Lactoferrin on Location Memory in Piglets

2.1. Location Memory Test

The location memory test was carried out on 36-day-old piglets. The piglets were allowed to familiarize themselves with the test area. The piglets in group 1 (control, n=16), group 2 (medium dose, n=17), and group 3 (high dose, n=18) went through the location memory test. In the test, four fixed partition boards (0.3×0.35×0.50 m) (FIG. 1) were placed in the front left (location 1), left corner (location 2), front central test area (location 3), and right back corners (location 4), which were 0.7, 0.8, and 0.9 m from the side walls for 1, 2 and 4 partition boards facing the door (FIG. 3). There were four bowls hidden by the four partition boards in the task zone, but only one bowl had accessible milk (FIG. 2B), while the milk in the other three bowls was inaccessible (FIG. 2A). The piglets could not see the bowls when they stayed in front of the partition boards. They had to go around to the back of the partition boards to find the accessible or inaccessible milk. The bowl with accessible milk was randomly changed in each trial. There were 10 trials for each piglet, including 4 trials in the morning, 4 trials in the afternoon, and 2 trials in the following morning. It was expected that “smarter” piglets would find the accessible milk more quickly without revisiting any inaccessible milk bowl. A mistake was registered when a piglet revisited the location of any inaccessible milk. The piglets' behavior in the test area was recorded by direct observation, and on videotapes for further analyses. All the tests were conducted by trained staff members.

Location memory was recorded when piglets entered the test zone and first visited the location where an accessible milk bowl was found in the previous trial (FIG. 4B). Two types of location memories were tested: short-term memory (STM) and long-term memory (LTM). In this study, we defined STM as memory for 2 consecutive trials with an interval of 5 min or less. All location memories lasting longer than 5 min were considered as LTM. There were three types of LTM in this study: (1) 30 min LTM (in the morning); (2) 4 h LTM (first trial in the afternoon); (3) 16 hours LTM (first trial the following morning). The schedule of location memory test is shown in FIG. 5.

2.2. Results

The overall difference in location memory between the groups is shown in Table 2. The medium dose and high dose groups had better location memory than the control group, but the difference did not reach statistical significance (P>0.05). However, when we analyzed data based on the number of success rate using total theoretic number of successes vs. the real number of successes made by piglets, the medium dose and high dose groups made 30% more successes than the control group (Table 2).

TABLE 2
Location memory success rate

| Group | N | Theoretic | Actual | Success rate¹ |
|---|---|---|---|---|
| Control | 16 | 128 | 18 | 14.1% |
| Medium dose | 17 | 136 | 28 | 20.6% |
| High dose | 18 | 144 | 30 | 20.1% |

¹Success rate = (actual number of successes/theoretic number of successes) × 100%

Mean number of successes in 9 location memory trials is shown in FIG. 8. Although the lactoferrin treatment groups performed better with regard to the location memory than the control group, the statistical analysis did not reach significance (P>0.05).

We also found that the lactoferrin treatment groups performed better with regard to the location memory than the control group when we considered the first 5 trials as acquisition phase learning and the last 5 trials as retrieval phase learning (FIG. 9).

Any location memory with an interval of no more than 5 min was defined as short-term location memory. Memories with an interval of more than 5 min were defined as long term location memories. The total number of successes of short-term and long-term location memory is summarized in Table 3.

TABLE 3
Short-term and long-term memory success rate

| Group | N | STM (5 min) | LTM (30 min) | LTM (16 h) |
|---|---|---|---|---|
| Control | 16 | 13/80 (16.3%)¹ | 1/32 (3.1%) | 4/16 (25.0%) |
| Medium dose | 17 | 17/85 (20.0%) | 4/34 (11.8%) | 7/17 (41.2%) |
| High dose | 18 | 16/90 (17.8%) | 7/36 (19.4%) | 7/18 (38.9%) |

¹Success rate = (actual number of successes/theoretic number of successes) × 100%.
P = 0.026

These results show that the groups treated with lactoferrin performed better both in the short-term and long-term memory tests. In particular, the high dose group showed a significantly better 30-min long-term memory than the control group (P=0.046) (FIG. 10).

Furthermore, the groups treated with lactoferrin performed better in the location memory trials than the control group, except for trials 5 and 6 (FIG. 11). In particular, in trial 7 the treated group performed significantly better than that the control group (P=0.026, two-way ANOVA).

### Example 3

### Effect of Lactoferrin on Learning Speed and Memory in Piglets

3.1. Learning and Memory Test

The 8-arm radial maze method [8] was used to test the cognitive functions of learning and memory capability. The piglets were introduced into the 8-arm radial maze individually. Two tests were carried out: an “easy task” (task 1) and a more “difficult task” (task 2) (FIG. 12). Both tests have accessible milk in one arm and inaccessible milk in the remaining 7 arms, so that all arms have the same smells to prevent the olfactory learning (FIG. 12). In both tests, a visual cue consisting of 3 black dots is placed randomly on a door with accessible milk (corresponding to their group milk) in the arm. In the easy task, one black dot visual cue is placed on the remaining 7 doors with inaccessible milk (the same amount and type of milk as the accessible milk). In the difficult task, a visual cue with 2 black dots is placed on the remaining 7 doors. The position of 3 black dots visual cue was changed between trials in a predetermined random order. Forty trials for each of task 1 and task 2 were conducted over a 10-day period, beginning on day 22 (22-day-old piglet).

Assessment of learning capacity was determined based on the number of trials taken to successfully learn the visual cue. Learning was quantified using the number of mistakes and successes in finding the accessible milk arm during each trial. A mistake was registered each time when the piglet entered or put its whole head through the wrong door. A success was registered when the piglet entered the correct door. The criterion of learned the visual cues were: (A) a maximum of 1 mistake in 3 consecutive trials. (B) no mistakes in 3 consecutive trials, (C) a maximum of 1 mistake in 4 consecutive trials, (D) no mistakes in 4 consecutive trials, (E) a maximum of 1 mistake in 5 consecutive trials (F) no mistakes across 5 consecutive trials. An overhead video camera recorded continuously during the learning and memory test, and a trained observer simultaneously recorded the results manually. All the tests were conducted by trained staff blinded to the level of lactoferrin intake. Results were corroborated by independent analysis of the video material. To reduce stress and familiarize the piglets with the test protocol, we allowed two piglets from the same pen into the maze to learn how to open and close the door before the learning test (8 trials).

There were 4 trials in the morning and 4 trials in the afternoon per day. Two consecutive trials were tested for one piglet. The intention interval time (change visual cue and place fresh milk) for two trials were 5 min (5 min short-term memory), the piglet was located at the waiting zone (outside test zone) during the 5 min period. Forty min later (40 min long-term memory), the piglet was introduced to radial maze again for 2 other consecutive trials. In the afternoon, the piglet repeated the morning session test. The intention interval between morning and afternoon test was 3 h (3 h long-term memory). The next day after 16 h, we repeated the same task as the previous day test (16 h long term memory). Total number of trial for easy and difficulty tasks were 40. Forty-eight hours after completion of the easy and difficult trials, piglets that have reached the learning criterion undertook the same pattern of task again as a ‘48 h long-term memory’. The number of mistakes in finding the accessible milk is recorded as an index of memory. The procedure of easy and difficult task arrangement is shown in FIG. 13.

In the easy learning task in FIG. 14, lactoferrin supplementation significantly improves learning speed when we consider the total number of mistakes in the first 20 trials (learning acquisition phase) as covariaes for analysis (P<0.05). In the difficult learning task, bLF supplementation significantly improves learning speed when we consider the total number of successes in all 40 trials (reinforcement) as covaries for analysis.

3.2 Results

The learning speed (based on the number of trials to learn the visual cues) in the medium dose group was the fastest compared to that in the high dose and control groups. When the number of mistakes per day was used as a measure of learning, the results showed that the piglets in the control group made more mistakes in the difficult task test. The difference between the groups was significant on day 4 (P<0.05, general linear model), which was attributed to a retrieval phase of the learning process. There was a trend in memory improvement in the groups treated with lactoferrin at the retention interval time of 5 min, 40 min and 3 h. The significant memory improvement was found at 40 min and 3 h, but not 16 h and 48 h. The results imply that the maximum retention interval time (capacity of memory) of recalling the visual cue was 3 h for the 38-day-old piglets. However there were not dose responses in memory test at different retention interval periods.

### Example 4

### BDNF Gene Expression in Hippocampus

Gene expression of brain-derived neutrotrophic factor (BDNF) was determined by subjecting hippocampus tissues from 38-day-old piglets to quantitative reverse transcription PCR analysis (qRT-PCR).

As shown in FIG. 15, lactoterrin supplementation significantly increased the relative mRNA levels of BDNF in hippocampus. Moreover, BDNF protein levels in hippocampus were significantly increased by medium dose lactoferrin supplementation (FIG. 16).

Thus, a correlation between behavior data and BDNF gene expression results were found suggesting that dietary lactoferrin likely functions by increasing the expression of BDNF.

### Example 5

### Expression of Key Genes in the BDNF Signaling Pathway

The expression of key genes in the BDNF signaling pathway was determined by subjecting hippocampus tissues from 38-day-old piglets to Affymetrix gene microarray analysis. The key genes that were analysed are listed in table 4.

As can be seen from the results shown in table 4, lactoferrin supplementation increased the expression of BDNF, GRB2, IRS1, Trk3, RAPGEF1(C3G), CAMK, MAPK11, MAPK12, CREB1, SP1, MYC, AND ESR1 in the hippocampus. These are all key genes in the BDNF signaling pathway. The BDNF signaling pathway is shown in FIG. 17.

As stated above, it is well established that activation of the BDNF signaling pathway leads to enhances phosphorolation and nuclear translocation of CREB. It is also established that the phosphorylation of CREB at serine 133 (pCREB) induces gene transcription, and plays a critical role in initiating learning and memory processes.

The Summary of genes involving BDNF neurotrophin signaling pathway were up- or down-regulated by dietary lactoferrin supplementation

TABLE 4

| | | | Fold- |
|---|---|---|---|
| | | | Change (compared |
| | | | with control |
| Gene Name | Probeset ID | p-value | group) |
| BDNF | Ssc.16243.1.S1_at | 0.004338 | 1.30597 |
| GRB2 | Ssc.27313.3.S1_at | 0.005372 | 1.07618 |
| IRS1 | Ssc.7304.2.A1_at | 0.005528 | 1.19099 |
| Trk3 | Ssc.4915.1.A1_at | 0.000455 | 1.24593 |
| PI3K | Ssc.11109.1.S1_at | 0.007745 | −1.17788 |
| RAP1A | Ssc.24315.1.S1_at | 0.002479 | −1.16274 |
| RAPGEF1(C3G) | Ssc.3567.1.S1_at | 0.041148 | 1.06101 |
| CAMK | Ssc.2491.1.S1_at | 0.009374 | 1.18927 |
| MAPK11 | Ssc.29722.1.S1_at | 0.034919 | 1.1143 |
| MAPK12 | Ssc.6498.1.A1_at | 0.002471 | 1.10944 |
| CREB1 | Ssc.8827.1.S1_at | 0.006465 | 1.2 |
| SP1 | Ssc.18559.1.S1_at | 0.000802 | 1.1819 |
| MYC | SscAffx.8.1.S1_at | 0.035227 | 1.16126 |
| ESR1 | gi: 52346219_at | 0.044021 | 1.17212 |

### Example 6

### pCREB Level in Hippocampus

The pCREB level was determined by subjecting hippocampus tissues from 38-day-old piglets to western blot analysis.

As shown in FIG. 18, lactoterrin supplementation significantly increased the level of pCREB in the hippocampus.

Based on the findings of examples 4 to 6, it is postulated the effect of lactoferrin supplementation on cognition and memory may, at least in part, stem from its positive effect on BDNF levels, and BDNF's subsequent effect on the BDNF's signaling transduction cascade and pCREB levels.

### Example 7

### PolySia-NCAM Level the Hippocampus and Pre-Frontal Cortex

The PolySia-NCAM level was determined by subjecting hippocampus and pre-frontal cortex tissue from 38-day-old piglets to western blot analysis.

As shown in FIG. 19 lactoterrin supplementation increased the level of PolySia-NCAM in the hippocampus and pre-frontal cortex.

Thus, a correlation between behavior data and PolySia-NCAM level was found suggesting that dietary lactoferrin may, at least in part, function by increasing polySia-NCAM levels. This indicates that the sialic acid moiety of lactoferrin may play a key role in increasing neuroplastisity and LTM.

A potential limitation for the quantitative assessment of the total level of polySia-NCAM that is increased by lactoferrin supplementation is that this glycan can form complexes with other neurotrophic factors, and therefore may be difficult to quantify by standard SDS-PAGE electrophoresis. Thus the findings disclosed herein of elevated levels of poly-Sia-NCAM in the hippocampus and pre-frontal cortex most likely represent the lower level of this glycan that is actually up-regulated by lactoferrin.

### REFERENCES

[1]. Fischer R, Debbabi H, Blais A, Dubarry M, Rautureau M, Boyaka P N, Tome D: Uptake of ingested bovine lactoferrin and its accumulation in adult mouse tissues. Int Immunopharmacol 2007, 7(10):1387-1393.
[2]. Ji B, Maeda J, Higuchi M, Inoue K, Akita H, Harashima H, Suhara T: Pharmacokinetics and brain uptake of lactoferrin in rats. Life Sci 2006, 78(8):851-855.
[3]. Lonnerdal B, Liao Y L, Du X G: miR-214 Regulates Lactoferrin Expression and Pro-Apoptotic Function in Mammary Epithelial Cells. J Nutr 2010, 140(9):1552-1556.
[4]. Roseanu A, Brock J H: What are the structure and the biological function of lactoferrin in human breast milk? Iubmb Life 2006, 58(4):235-237.
[5]. Rascon-Cruz Q, Gonzalez-Chavez S A, Arevalo-Gallegos S: Lactoferrin: structure, function and applications. Int J Antimicrob Ag 2009, 33(4).
[6]. Faldyna M, Adlerova L, Bartoskova A: Lactoferrin: a review. Vet Med-Czech 2008, 53(9):457-468.
[7]. Hettinga K, van Valenberg H, de Vries S, Boeren S, van Hooijdonk T, van Arendonk J, Vervoort J: The host defense proteome of human and bovine milk. PLoS One 2011, 6(4):e19433.
[8]. Wang B, Yu B, Karim M, Hu H, Sun Y, McGreevy P, Petocz P, Held S, Brand-Miller J: Dietary sialic acid supplementation improves learning and memory in piglets. Am J Clin Nutr 2007, 85(2):561-569.
[9]. Mikkelsen T L, Bakman S, Sorensen E S, Barkholt V, Frokiaer H: Sialic acid-containing milk proteins show differential immunomodulatory activities independent of sialic acid. J Agric Food Chem 2005, 53(20):7673-7680.
[10]. Kornum B R, Knudsen G M: Cognitive testing of pigs (Sus scrofa) in translational biobehavioral research. Neurosci Biobehav Rev 2011, 35(3):437-451.
[11]. Gieling E T, Nordquist R E, van der Staay F J: Assessing learning and memory in pigs. Animal Cognition 2011, 14(2):151-173.
[12]. Lind N M, Moustgaard A, Jelsing J, Vajta G, Cumming P, Hansen A K: The use of pigs in neuroscience: Modeling brain disorders. Neurosci Biobehav R 2007, 31(5):728-751.
[13]. Hernell O, Lonnerdal B: Iron status of infants fed low-iron formula: no effect of added bovine lactoferrin or nucleotides. Am J Clin Nutr 2002, 76(4):858-864.
[14]. Ryan P J, Ma S, Olucha-Bordonau F E, Gundlach A L: Nucleus incertus—an emerging modulatory role in arousal, stress and memory. Neurosci Biobehav Rev 2011, 35(6):1326-1341.
[15]. Schwabe L, Joels M, Roozendaal B, Wolf O T, Oitzl M S: Stress effects on memory: An update and integration. Neurosci Biobehav Rev 2011.
[16]. van Liempt S, Vermetten E, Lentjes E, Arends J, Westenberg H: Decreased nocturnal growth hormone secretion and sleep fragmentation in combat-related posttraumatic stress disorder; potential predictors of impaired memory consolidation. Psychoneuroendocrino 2011.

1. A method for improving memory and/or learning speed, and/or for promoting brain maturation in a healthy infant comprising the steps of administering lactoferrin to an infant.

2. The method according to claim 1, wherein the memory is spatial memory.

3. The method according to claim 1, wherein the memory is long-term memory.

4. The method according to claim 1, wherein the lactoferrin is administered to the healthy infant at a daily intake dose in the range of 100 to 400 mg/kg body wt/day.

5. The method according to claim 4, wherein the daily intake dose of lactoferrin is in the range of 100 to 200 mg/kg body wt/day.

6. The method according to claim 4, wherein the daily intake dose of lactoferrin is in the range of 250 to 350 mg/kg body wt/day.

7. The method according to claim 4, wherein the daily intake dose of lactoferrin is split up into at least two portions.

8. The method according to claim 1, wherein the lactoferrin is provided in an ingestible composition, selected from the group consisting of human food products, starter milks, growing up milks, infant feeding formulas, baby food and drinks, and maternal nutritional food.

9. The method according to claim 1, wherein the lactoferrin is present in a liquid ingestible composition at a concentration in the range of 0.1 to 2 g/l.

10. The method according to claim 1, wherein the lactoferrin is present in the liquid ingestible composition at a concentration in the range of 0.3 to 0.7 g/l.

11. The method according to claim 1, wherein the lactoferrin is present in the liquid ingestible composition at a concentration in the range of 0.8 to 1.2 g/l.

12. The method according to claim 1, wherein the lactoferrin is provided as a milk or whey fraction enriched in lactoferrin.
